(19) United States
(12) Patent Application Publication
(10) Pub. No.: US 2006/0177400 A1
(43) Pub. Date: Aug. 10, 2006

(54) Topical formulation for the treatment of urushiol induced contact dermatitis and method for treating the same

(76) Inventor: William M. Yarbrough, Peoria, IL (US)

Correspondence Address: King & Partners, PLC, 170 College Avenue, SUITE 230, HOLLAND, MI, 49423, US

(21) Appl. No.: 11/209,189

(22) Filed: Aug. 22, 2005

Related U.S. Application Data
(63) Continuation-in-part of application No. 10/200,691, filed on Jul. 22, 2002, now Pat. No. 7,008,963.
Application No. 10/200,691 is a continuation-in-part of application No. 09/347,714, filed on Jul. 3, 1999, now Pat. No. 6,423,746.

(51) Int. Cl. A61K 8/89; A61K 31/695

(52) U.S. Cl. 424/70.12; 514/63

(57) ABSTRACT

A topical formulation for the treatment of urushiol induced contact dermatitis, comprising: (1) a first medicament, wherein the first medicament is represented by the following chemical structure: 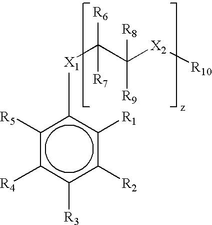
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 100; and
(2) a second medicament, wherein the second medicament is represented by the following chemical structure:

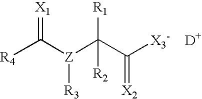

wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element, with the provisio that the first medicament and the second medicament cooperatively effectuate substantial removal of urushiol from an affected area of a patient having urushiol induced contact dermatitis.

### CROSS-REFERENCE TO RELATED APPLICATIONS

This application relates to U.S. application Ser. No. 10/200,691, filed Jul. 22, 2002, which is a continuation-in-part of U.S. application Ser. No. 09/347,714, filed Jul. 3, 1999, now U.S. Pat. No. 6,423,746 B1, all of which are hereby incorporated herein by reference in their entirety.

### BACKGROUND OF THE INVENTION

1. Field of the Invention

The present invention relates in general to topical formulations for treating urushiol induced contact dermatitis, and, more particularly, to a topical formulation for treating urushiol induced contact dermatitis which includes a first medicament and a second medicament, which cooperatively effectuate substantial removal of urushiol from an affected area of a patient having urushiol induced contact dermatitis.

2. Background Art

Urushiols are a group of compounds present in oils secreted by several plants of the Rhus genus (formerly Toxicodendron). The most notable members are poison oak, (Rhus diversilobum) and poison ivy (Rhus radicans), among others. The oils are typically secreted on the surface of the plant's leaves and are easily transferred to clothing and skin when the leaves are touched. However, urushiol in the root system and vines is pure and is approximately 10 to approximately 100 times more concentrated than that found in leaves. Accordingly, contact with cut or broken vines, or root systems will almost always result in a reaction. The urushiol, which is best characterized by the below identified structures, binds to or embeds in the skin cell surface and triggers a series of immunological responses that ultimately result in an itchy rash (i.e. Rhus dermatitis—a form of contact dermatitis). 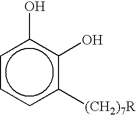 R═(CH₂)₇CH₃ I
R═HC═CH(CH₂)₅CH₃ II
R═HC═CHCH₂CH═CH(CH₂)₂CH₃ III
R═HC═CHCH₂CH═CHCH═CHCH₃ IV
R═HC═CHCH₂CH═CHCH₂CH═CH₂ V

As is shown above, urushiols are mixtures of catechols with long, hydrophobic, carbon (alkyl) side chains at the three position of the catechol ring. For example, poison ivy contains predominantly 3-n-pentadececylcatechols (C-15) and poison oak contains predominantly 3-n-heptaecylcatechols (C-17).

The American Academy of Dermatology estimates that there are up to 50 million cases of urushiol induced contact dermatitis annually in the United States alone. No one is sure of the number of world wide annual exposures, but some experts estimate that the number could be double that of the United States. Accordingly, urushiol induced contact dermatitis is a world wide problem.

Once urushiol touches the skin, it begins to penetrate in minutes. It is completely bound to or embedded in the skin after eight hours (two to six hours according to some experts). The rash generally develops within two days. Redness and swelling occur, often followed by blisters and severe itching. In a few days, the blisters may become crusted and begin to scale. The rash generally peaks after five days, and starts to decline after about a week to 10 days. The rash takes two or more weeks to heal. In a severe case or in a systemic reaction, individuals will many times have black spots in areas of heavy urushiol concentration.

The rash can affect almost any part of the body, especially where the skin is thin, such as on the face. A rash develops rarely on the soles of feet and palms of hands, where the skin is thicker. The rash does not spread, although is may seem to do so when it breaks out in new areas. This may happen because urushiol absorbs more slowly into skin that is thicker, such as on forearms, legs and the body's trunk. Urushiol can be transferred by fingernails or animal fur and can remain on clothing, shoes, and tools for up to five years in moist climates and nine years in dry climates.

The primary treatment for urushiol induced contact dermatitis has historically consisted of: (1) attempting to remove the oil as quickly after exposure as possible; (2) applying rubbing alcohol; (3) washing affected areas with water; and (4) showering with soap and water. In many instances, however, people either fail to fully remove the toxin before it has become bound to or embedded in the skin or do not realize they have been exposed until after the rash appears.

Pharmacies are replete with over-the-counter solutions. A partial list of the most popular solutions include hydrocortisone creams and ointments, diphenhydramine gels, calamine lotion, and a proprietary product, Ivy Dry®. Hydrocortisone, diphenhydramine, and calamine lotion are palliatives and offer only temporary relief from the itching associated with urushiol induced contact dermatitis, and they do nothing to remove urushiol from the skin. Likewise, Ivy Dry® provides essentially a cooling effect that is only temporary, and may, if used before the urushiol binds to the skin, remove son of the urushiol.

Attempts have been made to find both prophylactic solutions as well as post-exposure solutions. To date, no vaccine has been developed and the prior art solutions are not without shortcomings. One solution, is disclosed in U.S. Pat. No. 5,686,074, issued to Stewart, which teaches and claims a solution for poison ivy which includes a composition including linseed oil, an astringent, a starch, an essential oil and a citrus oil. One shortcoming of this patent is that linseed oil can cause irritation itself. A second shortcoming of this patent is that it requires that the composition be applied to the affected areas up to twice a day until the rash is gone. The composition provides what appears to be only very temporary palliative relief of poison ivy symptoms and does not appear to alter the course of the malady.

Other proposed solutions are disclosed in U.S. Pat. Nos. 5,620,527; 5,011,689; 4,499,086; 4,259,318; 4,002,737; 3,862,331; 3,875,301; and 3,922,342.

Yet other prior art attempts have focused on prophylactics for preventing the dermatitis. One example is disclosed in U.S. Pat. No. 4,663,151, issued to Waali which discloses and claims a prophylactic solution based upon Aluminum Chlorhydrate. Of course, the most significant shortcoming associated with prophylactic solutions is that they are only effective if applied before exposure to the urushiol—an occurrence that rarely takes place.

It is therefore an object of the present invention, to provide a topical formulation for the treatment of urushiol induced contact dermatitis which, among other things, remedies the aforementioned detriments and/or complications associated with use of “conventional” formulations and associated treatments for urushiol induced contact dermatitis.

### SUMMARY OF THE INVENTION

The present invention is directed to a topical formulation for the treatment of urushiol induced contact dermatitis comprising two or more micelle-forming surfactants, wherein micelle formation is generally enhanced and the formation of mixed micelles occurs. The formation of mixed micelles when an anionic surfactant or an amphoteric surfactant (a first medicament) is combined with a nonionic polyoxyethylene-based surfactant (a second medicament) is suggested for the reduction in skin irritation and increased effectiveness for removal of oils such as urushiols from the skin. Such is the case for one embodiment of the present invention which combines a mild anionic surfactant (sodium lauryl sarcosinate) with a nonionic polyoxyethylene-based surfactant (nonyl phenyl ethoxylate)—a/k/a nonoxynol-9. Examples of other suitable mild anionic surfactants include salts of sarcosinate, sulfoacetate, taurate and isethionate. Examples of somewhat less mild but nonetheless suitable anionic surfactants are salts of fatty acid carboxylates (soaps), sulfonates, sulfosuccinates, sulfates, and phosphoric acid mono- and di-esters. Suitable amphoteric surfactants include sultaines such as cocoamidopropylsultaine. Suitable nonionic polyoxyethylene-based surfactants also include octyl phenyl ethoxylate and PEG-alkylates.

The present invention is also directed to a topical formulation for the treatment of urushiol induced contact dermatitis comprising: (1) a first medicament, wherein the first medicament is represented by the following chemical structure: 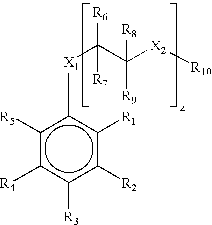
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof, wherein X₁₋₂ are the same or different and comprise O, S, or Se; and wherein z is an integer ranging from approximately 1 to approximately 100; and (2) a second medicament, wherein the second medicament is represented by the following chemical structure: 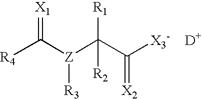
wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element, with the provisio that the first medicament and the second medicament cooperatively effectuate substantial removal of urushiol from an affected area of a patient having urushiol induced contact dermatitis.

In a preferred embodiment of the present invention, the first medicament is represented by the following chemical structure: 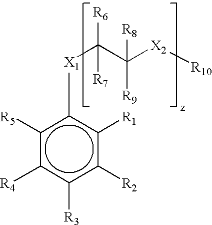
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 50.

In another preferred embodiment, the first medicament is represented by the following chemical structure:

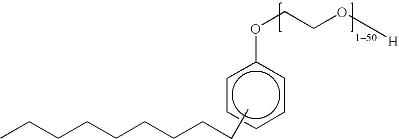

In this embodiment the first medicament may be represented by the following chemical structure: 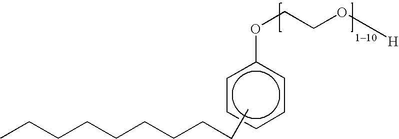
and comprise one or more ethoxylates, including, but not limited to, nonyl phenyl ethoxylate.

In yet another preferred embodiment of the present invention, the second medicament is represented by the following chemical structure: 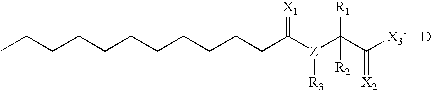
wherein R₁₋₃ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof, wherein X₁₋₃ are the same or different and comprise O, S, or Se; wherein Z comprises N or P; and wherein D comprises a Group I element.

Preferably, the second medicament is represented by the following chemical structure: 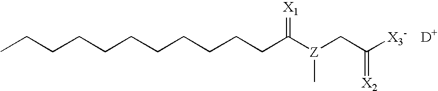
wherein X₁₋₃ are the same or different and comprise O, S, or Se; wherein Z comprises N or P; and wherein D comprises a Group I element.

In this embodiment, the second medicament may be represented by the following chemical structure: 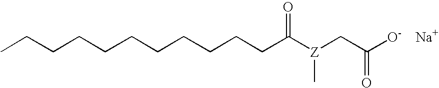
wherein Z comprises N or P; and comprise one or more sarcosinates, including, but not limited to, sodium lauryl sarcosinate.

In accordance with the present invention, the topical formulation may also comprise acetylated lanolin alcohol, a scrubbing agent, such as polyethylene granules, pumice, etcetera, and/or disodium EDTA.

The present invention is further directed to a method for treating urushiol induced contact dermatitis, comprising the steps of: (1) providing a patient, wherein the patient has an area affected by urushiol induced contact dermatitis; (2) providing a topical formulation comprising:
(a) a first medicament comprising an anionic and/or amphoteric surfactant, wherein the first medicament may be represented by the following chemical structure: 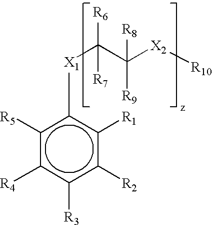
   wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 100; and
(b) a second medicament comprising a nonionic surfactant, wherein the second medicament may be represented by the following chemical structure: 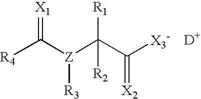
   wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element; (3) associating an effective amount of the topical formulation (having a mixed micelle) to the affected area of the patient; and (4) removing the topical formulation, and, in turn, effectuating substantial removal of urushiol from an affected area of a patient having urushiol induced contact dermatitis.

### BRIEF DESCRIPTION OF THE DRAWINGS

Not applicable

### DETAILED DESCRIPTION OF THE INVENTION

While this invention is susceptible of embodiment in many different forms, there will herein be described in detail several specific embodiments with the understanding that the present disclosure is to be considered as an exemplification of the principles of the invention and is not intended to limit the invention to the embodiments illustrated.

It will be understood that the chemical structures provided herein are merely schematic representations. As such, some of the bond lengths and bond angles have been distorted from their actual scale for pictorial clarity.

Without being bound to any particular theory, it has now been surprisingly discovered that the below-identified first and second medicaments cooperatively effectuate substantial removal of urushiol from an affected area of a patient having urushiol induced contact dermatitis in a synergistic manner. While the precise mechanism is unknown, it is believed that the first and second medicaments cooperate to rapidly displace the urushiol from the affected area of a patient, and then secondarily substantially preclude the urushiol from re-associating with the affected area through low level swelling of the epidermis which substantially seals the same from further irritation by the urushiol.

In a first embodiment of the present invention, a topical formulation for the treatment of urushiol induced contact dermatitis is disclosed, which comprises: (1) a first medicament, wherein the first medicament is represented by the following chemical structure: 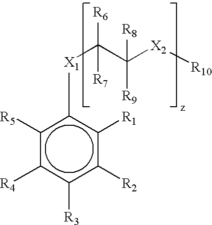
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 100; and (2) a second medicament, wherein the second medicament is represented by the following chemical structure: 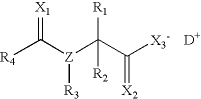
wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof, wherein X₁₋₃ are the same or different and comprise O, S, or Se; wherein Z comprises N or P; and wherein D comprises a Group I element.

In particular, the first medicament may be represented by the following chemical structure: 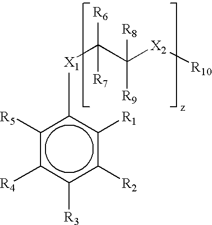
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof, wherein X₁₋₂ are the same or different and comprise O, S, or Se; and wherein z is an integer ranging from approximately 1 to approximately 100.

It will be understood that the nine carbon alkyl chain will be disclosed herein as delocalized because its position on the aromatic ring is mixed rather than locally defined at one position on the aromatic ring.

More specifically, the first medicament may be represented by the one of the following chemical structures:

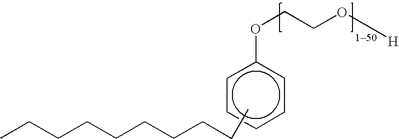

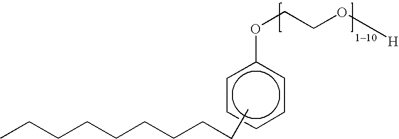

In a preferred embodiment of the present invention, the first medicament may comprise one or more ethoxylates, such as nonyl phenyl ethoxylate, which is commercially available from any one of a number of conventional chemical suppliers.

It will be understood that the above-identified compounds are commercially available from Aldrich Chemical Company of Milwaukee, Wis., among other common chemical vendors. While specific first medicament compounds have been disclosed, for illustrative purposes only, it will be understood that numerous other compounds that would have been obvious to one having ordinary skill in the art having the present disclosure before them are likewise contemplated for use.

In accordance with the present invention, the second medicament may be represented by the following chemical structure: 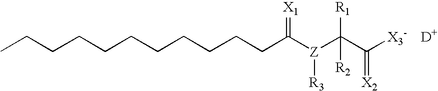
wherein R₁₋₃ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element.

More specifically, the second medicament may be represented by at least one of the following chemical structures: 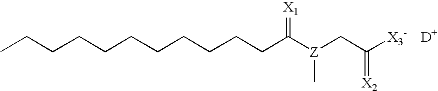
wherein X₁₋₃ are the same or different and comprise O, S, or Se; wherein Z comprises N or P; and wherein D comprises a Group I element; 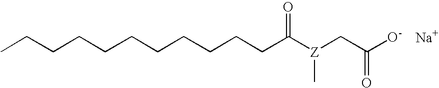
wherein Z comprises N or P.

In a preferred embodiment of the present invention, the second medicament may comprise one or more sarcosinates, such as sodium lauryl sarcosinate, which is commercially available from any one of a number of conventional chemical suppliers.

It will be understood that the above-identified compounds are commercially available from Aldrich Chemical Company of Milwaukee, Wis., among other common chemical vendors. While specific second medicament compounds have been disclosed, for illustrative purposes only, it will be understood that numerous other compounds that would have been obvious to one having ordinary skill in the art having the present disclosure before them are likewise contemplated for use.

Inert scrubbing agents can improve the action of the inventive composition. The scrubbing agent assists by causing the urushiol to detach from the skin, thereby rendering it more available for displacement. While any one of a number of scrubbing agents will enhance the topical formulation of the present invention, polyethylene granules appear to be superior. Another suitable inert agent includes pumice. The scrubbing agent should be large enough to be effective but not so large as to cause abrasions. The scrubbing agent should be in the range of 5 to 50 microns with an average size being approximately 25 microns or 50 mesh. It will be understood that while scrubbing agents are desirable, they do not appear to be a requisite to the operation of the present topical formulation.

To prepare the inventive composition, an exact ratio of the first medicament to the second medicament is not critical. However, in the case of nonyl phenyl ethoxylate to sodium lauryl sarcosinate, a weight ratio of approximately 1.5:5.0 to approximately 4.0:5.0 is preferred. The amount by weight of scrubbing agent can vary according to the grittiness desired. In the case of nonyl phenyl ethoxylate, sodium lauryl sarcosinate, and polyethylene beads, a weight ratio of about 40:20:40 is preferred. Thus, for production purposes, a formula having a first medicament ranging from approximately 20% to approximately 40% by weight, a second medicament ranging from approximately 10% to approximately 20% by weight, and a scrubbing agent from approximately 20% to approximately 50% by weight are reasonable. But again, the formula is not restricted to these ranges.

Moreover, a cutting agent that does not chemically react with the composition may be added, but is not required. The cutting agent makes the overall composition flow more easily, thereby enabling more packaging options, such as tubes. The cutting agent can be either an aqueous based solution or an oil based solution. The cutting agent must be added only in sufficient amount that it promotes flow but does not effect the action of the composition.

Thus, a representative composition for the present invention, among others, includes polyethylene granules (scrubbing agent), sodium lauryl sarcosinate (second medicament), nonoxynol-9 (first medicament), C12-15 pareth-9, disodium EDTA, quaternium-15, carbomer 2%, triethanolamine, and water—the commercial availability of which will be known to those having ordinary skill in the art inasmuch as the above-identified materials, and derivatives thereof, are commercially available throughout the chemical industry.

In use, the topical formulation is applied to an affected area of a patient and worked over the area by a scrubbing motion. After sufficient time has elapsed to ensure that the affected area has been adequately exposed to the topical formulation, typically thirty seconds to three minutes depending on the agents used, severity of the reaction, and the sensitivity of the individual, the topical formulation urushiol are washed away. To have the greatest effect, the topical formulation can be left on the affected area, after the initial scrubbing, for a short time, thirty to sixty seconds, a second rubbing can be accomplished. The second rubbing period can be shorter than the initial period and than washed away. Experiments have demonstrated that a majority of people need only one application to be relieved of itching; however, severe or systemic cases may require two or more applications approximately eight hours apart for two days to be fully effective. The inventive composition works at varying rates of effectiveness at any time during the rash cycle.

The foregoing description merely explains and illustrates the invention and the invention is not limited thereto except insofar as the appended claims are so limited, as those skilled in the art who have the disclosure before them will be able to make modifications without departing the scope of the invention.

What is claimed is:

1. A topical formulation for the treatment of urushiol induced contact dermatitis, comprising:.
a first medicament, wherein the first medicament is represented by the following chemical structure: 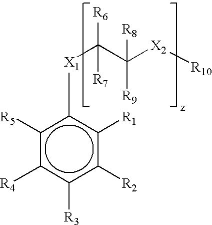
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 100; and
a second medicament, wherein the second medicament is represented by the following chemical structure: 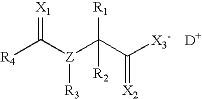
wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element, with the provisio that the first medicament and the second medicament cooperatively effectuate substantial removal of urushiol from an affected area of a patient having urushiol induced contact dermatitis.

2. The topical formulation according to claim 1, wherein the first medicament is represented by the following chemical structure: 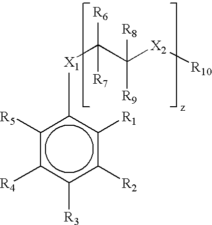
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 50.

3. The topical formulation according to claim 1, wherein the first medicament is represented by the following chemical structure:

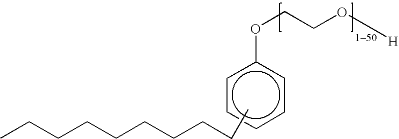

4. The topical formulation according to claim 1, wherein the first medicament is represented by the following chemical structure:

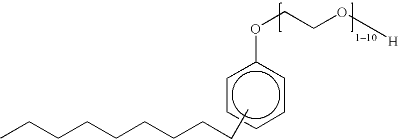

5. The topical formulation according to claim 1, wherein the first medicament comprises at least one ethoxylate.

6. The topical formulation according to claim 1, wherein the first medicament comprises nonyl phenyl ethoxylate.

7. The topical formulation according to claim 1, wherein the second medicament is represented by the following chemical structure: 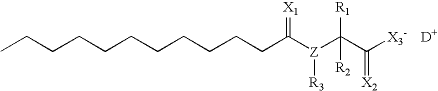
wherein R₁₋₃ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element.

8. The topical formulation according to claim 1, wherein the second medicament is represented by the following chemical structure: 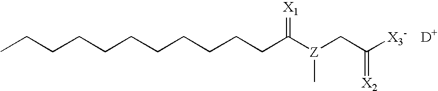
wherein X₁₋₃ are the same or different and comprise O, S, or Se; wherein Z comprises N or P; and wherein D comprises a Group I element.

9. The topical formulation according to claim 1, wherein the second medicament is represented by the following chemical structure: 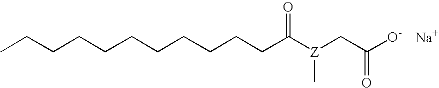
wherein Z comprises N or P.

10. The topical formulation according to claim 1, wherein the second medicament comprises at least one sarcosinate.

11. The topical formulation according to claim 1, wherein the second medicament comprises sodium lauryl sarcosinate.

12. The topical formulation according to claim 1, further comprising acetylated lanolin alcohol.

13. The topical formulation according to claim 1, further comprising a scrubbing agent.

14. The topical formulation according to claim 12, wherein the scrubbing agent is selected from the group consisting of polyethylene granules, pumice, and mixtures thereof.

15. The topical formulation according to claim 1, further comprising disodium EDTA.

16. A method for treating urushiol induced contact dermatitis, comprising the steps of:
providing a patient, wherein the patient has an area affected by urushiol induced contact dermatitis;
providing a topical formulation comprising:
a first medicament, wherein the first medicament is represented by the following chemical structure: 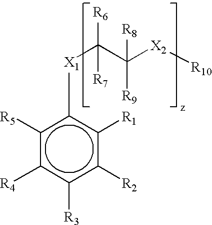
wherein R₁₋₁₀ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₂ are the same or different and comprise O, S, or Se; and wherein z is an integer ranging from approximately 1 to approximately 100; and
a second medicament, wherein the second medicament is represented by the following chemical structure: 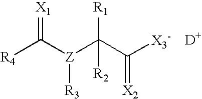
wherein R₁₋₄ are the same or different and comprise H, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof; wherein X₁₋₃ are the same or different and comprise O, S, or Se; wherein Z comprises N or P; and wherein D comprises a Group I element;
associating an effective amount of the topical formulation to the affected area of the patient; and
removing the topical formulation, and, in turn, effectuating substantial removal of urushiol from an affected area of a patient having urushiol induced contact dermatitis.
